(19) United States
(12) Patent Application Publication
(10) Pub. No.: US 2011/0189289 A1
(43) Pub. Date: Aug. 4, 2011

(54) Cell-Based Method and Means for Rebuilding Bone

(75) Inventors: Bernard Hoflack, Dresden (DE); Maria Arantzazu Sanchez Fernadez, Dresden (DE)

(73) Assignee: TECHNISCHE UNIVERSITAET DRESDEN, Dresden (DE)

(21) Appl. No.: 13/057,482

(22) PCT Filed: Aug. 4, 2009
(86) PCT No.: PCT/EP2009/060078

§ 371 Date: Apr. 22, 2011

(30) Foreign Application Priority Data
Aug. 4, 2008 (DE) 10 2008 036 060.0

(51) Int. Cl. A61K 9/14; A61K 35/12; A61P 19/00

(52) U.S. Cl. 424/484; 424/93.7

(57) ABSTRACT

The invention relates to the use of in vitro isolated or in vitro differentiated osteoclasts for bone rebuilding, in particular by in vivo application, to trigger bone rebuilding by attracting osteoblasts and preferably also vascular cells to sites of bone defect. It has been found by the inventors that osteoclasts can control bone rebuilding by secreting chemotactic factors and attracting osteoblasts and vascular cells to a site of bone defect. The invention further relates to the use of osteoclasts for the manufacture of a pharmaceutical preparation or medical product containing osteoclasts for the use for bone rebuilding.

The invention concerns the field of medicine, in particular the fields of surgery and orthopedics, by providing a cell-based method and means for rebuilding bone.

Bones form the major structural support of the body. They obtain their stability mainly through the osseous tissue, which they consist of Bones undergo remodeling to maintain the mass, the shape and the physical properties of the skeleton. Two major cell types, the bone-forming osteoblasts and the bone-resorbing osteoclasts contribute to this process, which occurs continuously throughout life. Bone remodeling relies on the tightly regulated interplay between bone forming osteoblasts and bone digesting osteoclasts.

The balance between bone formation and degradation is normally tightly controlled but it becomes deregulated, shifting towards more degradation under pathological conditions or during aging, thereby leading to osteoporosis (Teitelbaum S. L. (2000) Bone resorption by osteoclasts. Science 289: 1504-1508; Harada S. and Rodan G. A. (2003) Control of osteoblast function and regulation of bone mass. Nature 423: 349-355; Aguila H. L. and Rowe D. W. (2005) Skeletal development, bone remodeling, and hematopoiesis. Immunol Rev 208: 7-18.). This tight balance implies the existence of mechanisms coordinating the differentiation of osteoblasts and osteoclasts as well as their migration to locations where they function.

Osteoblasts control the differentiation of hematopoietic osteoclast precursors towards mature multinucleated cells, i.e. osteoclastogenesis (Boyle W. J. et al. (2003) Osteoclast differentiation and activation. Nature 423: 337-342). In combination with stromal cells they control bone degradation by expressing the Macrophage-Colony Stimulating Factor (M-CSF) required for the proliferation of osteoclast precursors and the Receptor for Activation of NF-KB Ligand (RANKL), a TNF family member triggering their differentiation (Teitelbaum S. L. (2000) Bone resorption by osteoclasts. Science 289: 1504-1508; Boyle W. J. et al. (2003) Osteoclast differentiation and activation. Nature 423: 337-342; Wagner E. F. (2002) Functions of AP1 (Fos/Jun) in bone development. Ann Rheum Dis 61 Suppl 2: ii40-42; Kawamata A. et al. (2008) JunD suppresses bone formation and contributes to low bone mass induced by estrogen depletion. J Cell Biochem 103: 1037-1045; Bruzzaniti A. and Baron R. (2006) Molecular regulation of osteoclast activity. Rev Endocr Metab Disord 7: 123-139; Teitelbaum S. L. (2007) Osteoclasts: what do they do and how do they do it? Am J Pathol 170: 427-435).

There is also evidence, that osteoclasts can control osteoblast function. Ephrin B2 expressed by osteoclasts and its Ephrin B4 receptor expressed by osteoblasts allow bidirectional signaling (Zhao C. et al. (2006) Bidirectional ephrinB2-EphB4 signaling controls bone homeostasis. Cell Metab 4: 111-121). Signaling through Ephrin B4 into osteoblasts enhances osteogenic differentiation whereas signaling through Ephrin B2 into osteoclast precursors suppresses osteoclast differentiation. It has also been reported that the v-ATPase V₀ subunit D2 is not only involved in osteoclast fusion but also regulates the secretion by osteoclasts of still unidentified factors that inhibit the differentiation of osteoblast precursors into mature cells (Lee S. H. et al. (2006) v-ATPase V0 subunit d2-deficient mice exhibit impaired osteoclast fusion and increased bone formation. Nat Med 12: 1403-1409).

During development, osteoblasts must colonize the cartilage that will be replaced by bone. During adulthood, bone remodeling and repair require the migration of osteoblasts to bone areas that need to be rebuilt. This latter process also requires the mobilization of their progenitors residing together with MSCs (Mesenchymal stem cells) into niches of the bone marrow (Li L. and Xie T. (2005) Stem cell niche: structure and function. Annu Rev Cell Dev Biol 21: 605-631; Yin T. and Li L. (2006) The stem cell niches in bone. J Clin Invest 116: 1195-1201). A plethora of growth factors used as recombinant proteins, in particular BMPs, platelet-derived growth factor (PDGF), vascular endothelial growth factor (VEGF) and leukemia inhibitory factor (LIF), have been shown to exhibit in vitro chemotactic activities towards various cell types including osteoblasts and their progenitors (Fiedler J. et al. (2004) To go or not to go: Migration of human mesenchymal progenitor cells stimulated by isoforms of PDGF. J Cell Biochem 93: 990-998; Mayr-Wohlfart U. et al. (2002) Vascular endothelial growth factor stimulates chemotactic migration of primary human osteoblasts. Bone 30: 472-477).

Several growths factors, such as the Platelet derived Growth Factor PDGF or the vascular endothelial growth factor VEGF, acting as chemo-attractants of osteoblasts and endothelial cells respectively, are known to favor the bone rebuilding process. Some others are key factors regulating skeleton development, such as the bone morphogenic proteins BMPs. Therefore, these growth factors have been used to further improve the properties and the functionality of biomaterials. Some others have also been applied as soluble components in bone surgery. BMPs, available as recombinant proteins, are registered for different clinical applications. The OP1-Factor is registered for orthopedic applications and BMP2 for oral, maxillofacial and implantological applications in the USA since March 2007; 17 years after the discovery of these proteins. There are other preparations containing recombinant BMP2 and BMP7, with osteo-inductive effect (InductOs, INFUSEbonagraft, Osigraft). Thus, the use of osteoclasts as a source of growth factors favoring bone rebuilding would provide an alternative solution.

These growth factors have potential to be used to propagate the regrowth/rebuilding/remodeling of bone in a patient. There are many influences on bone that can make such a rebuilding of bone necessary. Bones can break as a result of mechanical stress, leading to fractures. Primary malignant bone tumors like sarcomas (Osteosarcome, Ewing-Sarcome) or secondary metastases originating from tumors in other tissues can destroy bone tissue (osteolysis) and often need to be removed surgically. Infection of bone tissue by bacteria, viruses or fungi can lead to osteomyelitis and to subsequent destruction of bone. Extraction or loss of teeth can cause the jaw bone to recede which impedes the fixation of implants or dentures.

However, after an initial hope, several recombinant growth factors turned out to be poor inductors of bone growth. VEGF is one example that is inefficient in bone rebuilding, most likely due to the chemical methods used absorb these growth factors and to “functionalize” the biomaterials. Beside the technical and scientific issues concerning the application of such recombinant proteins, the high cost remains the major hurdle to widely spread clinical applications.

The bone-building osteoblasts can up to date only be obtained in vitro by isolation of mesenchymal stem cells from bone marrow by a major surgical procedure. This puts an additional strain on the patient who may already be suffering from the implications of a serious disease such as a bone tumor.

The problem is probably to target the growth factors and cells to those places where they are needed.

It is still unclear which cell types in the bone matrix secrete the factors able to attract bone rebuilding cells, which growth factors can function in this context and how the migration of bone rebuilding cells is coordinated with bone digestion.

The identification of such cells and the factors they use is highly desirable because they could be used to attract osteoblasts to sites where rebuilding of bone material is required, e.g. bone damage, bone fractures, etc.

Bone diseases are emerging as one of the major health problems in our developed societies. There are roughly one million cases of skeletal defects per year requiring bone-grafts, which represent major challenges in bone surgery applicable to several medical disciplines. For example, two major types of cancers (breast and prostate) occurring in our populations lead to bone metastasis and fractures of affected bones. Millions of fractures per year due to osteoporosis have been reported worldwide and demographic changes predict a dramatic increase in osteoporotic fractures.

Thus, there is still a need to develop novel methods for rebuilding bone.

It is thus the aim of this invention to provide a method and means for rebuilding bone.

Surprisingly it has been found by the inventors that osteoclasts can control bone rebuilding by secreting chemotactic factors and attracting osteoblasts and vascular cells to a site of bone defect. The inventors now have found that mature osteoclasts, but not their precursors, secrete several growth factors that can be involved in bone rebuilding and/or bone vascularization. The growth factors that are secreted by mature osteoclasts regulate osteoblast chemotaxis and thus attract the osteoblasts and vascular cells to a site of bone defect. In particular it has been found that mature osteoclasts secrete PDGF-bb, which is recognized by PD3F/PDGF receptor beta (PDGFR-beta) on the surface of osteoblasts and regulates osteoblast chemotaxis.

The problem is thus solved by the use of in vitro isolated or in vitro differentiated osteoclasts for bone rebuilding, in particular by in vivo application, to trigger bone rebuilding by attracting osteoblasts and preferably also vascular cells to sites of bone defect. Another object is the use of osteoclasts to manufacture a pharmaceutical preparation or medical product for bone rebuilding or the treatment of bone defects.

The osteoclasts are used in the invention as natural factories to produce chemotactic factors or growth factors acting on osteoblasts or their precursors (mesenchymal stromal cells) as well as other cell types, will favor their chemo attraction to the place where bone needs to be repaired. Therefore, it will not be necessary to produce recombinant growth factors themselves.

Bone rebuilding herein refers to the formation of new bone material. The term bone rebuilding includes in particular remodeling, repair and regeneration of bone, which are necessary in case of fractures (including fissures, as well as osteoporotic fractures), skeletal defects, loss of bone due to cancer, surgery, bone diseases or due to other causes. The term also includes bone remodeling in case of bone atrophy. One example is the atrophy of the jaw-bone after the loss of one or several teeth.

Osteoclasts have been known to be involved in the degradation of bone before, but it has never been described in the state of the art that they can function as a means for the formation of new bone material and that this is due to their capability to secrete factors which are able to attract the bone rebuilding osteoblasts to sites where bone has to be rebuilt.

The term bone defect includes any bone with a damage or at least partial loss of function, not only including fractures, but also shrunk or atrophic bone.

The term osteoclast according to the invention includes in vitro isolated cells, cell lines and most preferably in vitro differentiated cells.

The use of osteoclasts advantageously opens up a simple, non-invasive way to obtain cells which can be used for bone rebuilding.

In contrast to osteoblasts, osteoclasts can easily be obtained by in vitro differentiation starting with a blood probe, in particular by isolation of mononuclear cells, preferably monocytes like macrophages, from the patient's blood. Using monocytes, osteoclasts can easily be obtained in vitro by adding macrophage colony stimulating factor (M-CSF) and receptor for activation of NF-κB ligand (RANKL) to the cell culture medium.

M-CSF controls the proliferation of osteoclast precursors (monocytes, macrophages) whereas the receptor for activation of NF-κB ligand (RANKL) controls their differentiation into mature, functional osteoclasts. The differentiation process can now be rapidly reproduced in vitro using recombinant MCSF and RANKL.

Obtaining the blood sample is done quickly and without major stress to the patient, and 500 ml blood are enough to obtain 5-10 mg osteoclasts within less than five days.

Thus a preferred object of the invention is the use of osteoclasts whereas the osteoclasts are obtained starting from isolated autologous precursor cells, thus cells which are isolated a few days before from the patient to be treated. These precursor cells are preferably mononuclear cells which have been obtained from a blood sample of that patient. To harvest the cells, blood is taken from a donor's vein (from a peripheral vein or via central venous catheter) and mononuclear cells are isolated by standard procedures—which can even be done via aphaeresis. The isolation via aphaeresis has the advantage that red blood cells and plasma can be returned to the donor.

The invention thus advantageously provides an autologous cell-based strategy for bone rebuilding, which is non-invasive. The concept underlying the invention is to activate and to recruit the endogenous bone repair system of the patient body to a site of bone defect.

In the inventive use the osteoclasts are administered locally to the patient. They are preferably administered in proximity to the bone defect, meaning directly to the site of bone defect or nearby. As the function of the osteoclasts according to the invention is to recruit other cells, in particular osteoblasts and/or their precursors, it is advantageously not necessary to apply the osteoclasts exactly in the area of bone defect (also herein referred to as site of bone defect). The recruitment to the site of bone defect also functions in case the osteoclasts are administered nearby.

Surprisingly, the osteoclasts do not need any crosstalk with other cells to secrete chemotactic factors and growth factors to attract osteoblasts and preferably also vascular cells to the site of the bone defect. Thus, according to the invention, the osteoclasts can be applied without adding any other cells. In particular the addition of osteoblasts is not necessary in the invention (as they are recruited in vivo). Also the addition of recombinant growth factors or chemokines is not necessary in the osteoclasts preparation. Consequently, the osteoclasts preparation preferably does not contain recombinant growth factors or chemokines However, it might contain traces of RANKL and M-CSF used to obtain the mature osteoclasts.

The invention comprises also a pharmaceutical preparation or medical product containing osteoclasts for the use for bone rebuilding. The invention further comprises the use of osteoclasts in the manufacture of a medicament for bone rebuilding and/or the treatment of bone defects.

The osteoclasts are preferably administered in form of suspensions or embedded in gels (like a hydrogel or xerogel) or bone cements or even in an implant, that are used to reconstruct missing bone.

The invention further comprises a method for the treatment of a subject, preferably a mammal, with a bone defect, with the steps:

a.) isolation of blood from the subject and enrichment of monocytes,

b.) differentiation of monocytes into osteoclasts (as described above),

c.) applying the osteoclasts obtained in step b.) to the subject in or nearby the area of the bone defect.

The osteoclasts applied in step c.) attract osteoblasts and vascular cells to the site of bone defect. The osteoblasts and vascular cells then rebuild the bone matrix.

The invention is further illustrated by the following figures and examples without being restricted to them.

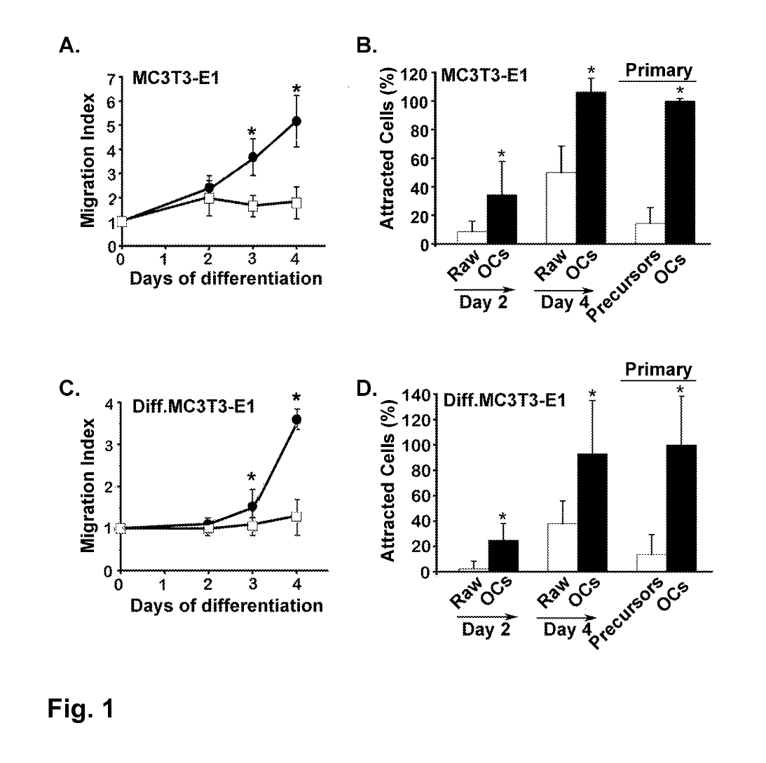

FIG. 1 shows the chemotaxis response of osteoblasts and osteoblast precursors to factors secreted by osteoclasts. Migration Index of mouse pre-osteoblastic MC3T3-E1 cells (A) and derived osteoblasts (diff MC3T3-E1) (C) in response to conditioned media of Raw264.7 cells (white squares or columns) and derived osteoclasts (black circles or columns). Chemotaxis of pre-osteoblastic MC3T3-E1 cells (B) and derived osteoblasts (D) by conditioned media of primary osteoclasts and their precursors. For comparison, the chemotactic activity of conditioned media from Raw264.7 cells and derived osteoclasts collected after 2 and 4 days of differentiation are shown. Shown are the mean values ±S.D. of four independent experiments performed in triplicates. P values from ANOVA tests equal or less than 0.05 were considered significant and are marked with an asterisk (*).

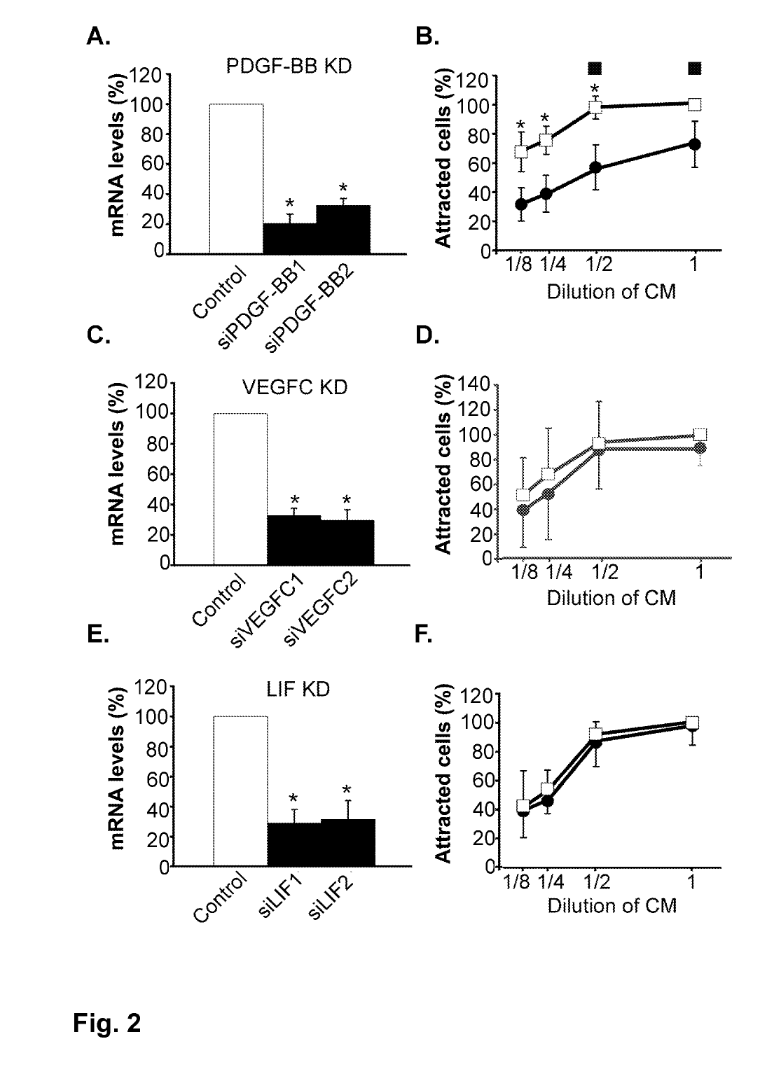

FIG. 2 shows that PDGF-bb secreted by osteoclasts triggers osteoblast chemotaxis.

Knockdown efficiencies of PDGF-bb (A), VEGFc (C) and LIF (E) in Raw264.7 cell-derived osteoclasts were determined by quantitative RT-PCR. The knockdown efficiencies were 74%, 71% and 70% respectively (p<0,00001, ANOVA). Chemoattraction of pre-osteoblastic MC3T3 cells by different dilutions of conditioned media of Raw264.7 cell derived osteoclasts treated with control siRNAs (white squares) or Raw264.7 cell-derived osteoclasts (black circles) in which the expression of PDGF-bb (B), VEGFc (D) and LIF (F) were silenced (p<0,0001, ANOVA). ▪ in B shows the rescue of PDGF-bb knockdown in osteoclasts: conditioned media of siRNA-treated osteoclasts were supplemented with 10 ng/ml recombinant human PDGF-bb. Data points represent the average of 5 experiments.

### EXAMPLE 1

Osteoclasts secrete factors attracting osteoblasts.

All cell lines were from ATCC (Rockville, Md., USA). Mouse pre-osteoblastic MC3T3-E1 cells were maintained in a-MEM supplemented with 10% heat inactivated Fetal Calf Serum (FCS). Mouse myeloid Raw264.7 cells were cultured in high glucose DMEM supplemented with 10% heat inactivated FCS. Primary osteoclast precursors were obtained from bone marrow of long bones of 8 week-old C57BL/6J mice. After purification on density gradients (Eurobio), they were cultured in α-MEM supplemented with 10% heat inactivated FCS. Soluble recombinant RANKL was from Abcys (Paris, France) or produced in Pichia yeast as described previously (Czupalla C. et al. (2006) Proteomic analysis of lysosomal acid hydrolases secreted by osteoclasts: implications for lytic enzyme transport and bone metabolism. Mol Cell Proteomics 5: 134-143). Recombinant human PDGF-bb was from PeproTech EC (London, UK), human recombinant M-CSF from Prospec Tany TechnoGene (Rehovot, Israel).

To show that osteoclasts can signal to osteoblasts in vitro, cell systems of osteoclastogenesis or osteoblastogenesis were used. First, mouse monocyte/macrophage-like Raw264.7 cells were stimulated with the osteoclastogenic cytokine RANKL to differentiate in vitro towards mature osteoclasts as described (Czupalla C. et al. 2006). Primary osteoclast precursors from mouse bone marrow and derived osteoclasts, which were obtained by differentiating in the presence of M-CSF and RANKL (Bonnelye E. et al. (2008) Dual effect of strontium ranelate: stimulation of osteoblast differentiation and inhibition of osteoclast formation and resorption in vitro. Bone 42: 129-138), were also used.

The conditioned media of osteoclasts were collected every 24 h, centrifuged, buffered with 20 mM HEPES pH 7.2 and kept at −80° C. until further use.

Second, mouse pre-osteoblastic MC3T3-E1 cells were used that differentiate in vitro towards mature osteoblasts upon stimulation with chemical cocktails containing 10⁻⁷M dexamethasone, 50 μg/m1 ascorbic acid and 10 mM β-glycerophosphate in α-MEM for ≈15 days.

To test whether the conditioned medium of Raw264.7 cells or Raw264.7-derived osteoclasts could attract pre-osteoblastic MC3T3-E1 cells or MC3T3-E1-cell derived osteoblasts, the Boyden chamber assay was used to measure chemotaxis. FIG. 1A shows that the chemotactic activity of Raw264.7 cells towards MC3T3-E1 cells was rather low. However, their chemotactic activity increased with time when they were differentiated towards osteoclasts upon stimulation with RANKL.

After 4 days of RANKL-induced differentiation, the conditioned medium of Raw-derived osteoclasts exhibited a clear chemotactic activity towards MC3T3-E1 cells as shown by Migration Index of mouse pre-osteoblastic MC3T3-E1 cells (FIG. 1A) in response to conditioned media of Raw264.7 cells (white squares) and derived osteoclasts (black circles). This activity was also observed with the conditioned medium of osteoclasts derived from bone marrow progenitors stimulated by M-CSF and RANKL as shown by the chemotaxis of pre-osteoblastic MC3T3-E1 cells (FIG. 1B) by conditioned media of primary osteoclasts and their precursors.

The conditioned medium of Raw264.7-derived osteoclasts also exhibited a chemotactic activity towards MC3T3-E1-derived osteoblasts, although to a lower extent, as shown by the Migration Index of derived osteoblasts (diff MC3T3-E1) (C) in response to conditioned media of Raw264.7 cells (white squares) and derived osteoclasts (black circles) (FIG. 1C). The chemotactic index of mature osteoclast conditioned media was two fold higher towards pre-osteoblastic MC3T3-E1 cells when compared to MC3T3-E1 cell-derived osteoblasts. Similar results were obtained with the conditioned media of primary osteoclasts derived from bone marrow progenitors (FIG. 1D).

These results confirm that, during their differentiation osteoclasts derived from Raw264.7 cells or primary bone marrow progenitors acquire the capability of secreting chemotactic factors able to attract osteoblast precursors and, to a lower extent, mature osteoblasts.

### EXAMPLE 2

PDGF-bb secreted by osteoclasts mediates osteoblast chemotaxis.

A siRNA-based strategy was used to identify the chemotactic factor(s) secreted by mature osteoclasts. Raw264.7-derived osteoclasts were first electroporated in the presence of siRNA probes. For this Raw 264.7 cells were differentiated into osteoclasts in the presence of RANKL. After 2-3 days, they were detached by incubation in PBS containing 0,5 mM EDTA. Pre-designed stealth RNAi or scrambled stealth RNAi duplexes were electroporated into osteoclasts. Electroporated cells were resuspended in medium supplemented with RANKL and maintained in culture for 48 h.

Conditioned media were collected and osteoclasts were processed for total RNA isolation and protein determination. Mouse preosteoblastic MC3T3-E1 cells or chemically differentiated osteoblasts were transfected with Stealth siRNA duplex oligonucleotides using Interferin as transfection reagent. After 48 h, the cells were harvested and the silencing efficiencies were determined by quantitative RT-PCR. The total RNAs were isolated and DNase I-treated RNAs were reverse transcribed. Quantitative RT-PCR was performed with a Stratagene Mx4000 QPCR system and the Brilliant SYBR Green QPCR kit according to the manufacturer's instructions (Stratagene, La Jolla, Calif.). Quantitative RT-PCR analyses were performed in triplicates, and Ct values were normalized using GAPDH.

The conditioned media of siRNA-treated osteoclasts were tested for their chemotactic activity. Chemotactic responses were measured in triplicates using a 48-well Boyden microchemotactic chamber (Falk W. et al. (1980) A 48-well micro chemotaxis assembly for rapid and accurate measurement of leukocyte migration. J Immunol Methods 33: 239-247). The lower wells of the apparatus were filled with growth factors in α-MEM containing 20 mM HEPES pH 7,2 or conditioned medium from Raw 264.7 cells or derived-osteoclasts and overlaid with a polycarbonate membrane of 5 μm pores (NeuroProbe Inc. Gaithersburg Md., USA). Cells (0.35×10⁵ MC3T3-E1, 0.45×10⁵ differentiated osteoblasts or 0.25x10⁵ 7F2 cells) in 50 μl of α-MEM were added to the upper wells. After a 3,5 h incubation at 37° C., the membrane was removed. The cells on the upper surface were discarded by gentle scraping and cells that had migrated to the other side of the membrane were fixed with 3% Paraformaldehyde, stained with toluidine blue (Sigma, Germany) and counted. The chemotactic index (CI) represents the ratio between the average number of cells migrating under given conditions and the average number of cells migrating under control conditions.

As shown in FIG. 2, an efficient siRNA-mediated depletion of PDGF-bb, VEGFc or LIF (≈80%) could be achieved in Raw-derived osteoclasts.

A reduction in PDGF-bb expression in Raw-derived osteoclasts resulted in a ≈50% reduction in the ability of their conditioned medium to attract pre-osteoblastic MC3T3-E1 cells at every concentration tested. The residual chemotactic activity of pre-osteoblastic MC3T3-E1 cells by conditioned media of siRNA-treated osteoclasts most likely reflects the presence of low amounts of PDGF-bb still secreted by these cells.

In addition, this loss of chemotactic activity after PDGF-bb knockdown could be rescued by the addition of recombinant PDGF-bb, reaching the same chemotactic activity of conditioned media of non-siRNA-treated osteoclasts (FIG. 2B). In contrast, an 80% reduction in VEGFc or LIF expression remained without any effect on chemotaxis of MC3T3-E1 cells (FIG. 2C-F). Because recombinant CCL9, IL1ra and Twgs1 did not modify the chemotactic activity of MC3T3-E1 cells (data not shown), they were not further considered. We conclude from these results that PDGF-bb secreted by Raw264.7-derived osteoclasts acts as a potent chemotactic agent towards preosteoblastic MC3T3-E1 cells.

What is claimed is:

1.-10. (canceled)

11. A method of rebuilding bone, comprising the step of administering to a mammal osteoclasts that have been isolated in vitro or differentiated in vitro.

12. The method according to claim 11, wherein the osteoclasts are administered to the mammal in vivo in proximity to a site of a bone defect.

13. The method according to claim 12, comprising the steps of isolating precursor cells from a patient, differentiating in vitro the precursor cells to the osteoclasts, and administering the osteoclasts to said patient in proximity to the site of the bone defect.

14. The method according to claim 13, wherein the precursor cells are monocytes derived from peripheral blood of said patient.

15. The method according to claim 11, wherein the osteoclasts attract osteoblasts and/or vascular cells to the site of the bone defect.

16. The method according to claim 11, wherein the bone defect is a fracture, skeletal defect or loss of bone due to atrophy, bone disease, cancer, surgery or other causes.

17. The method according to claim 11, comprising the step of preparing a suspension containing the osteoclasts.

18. The method according to claim 11, comprising the step of embedding the ostecoclasts in a gel or in a bone cement or an implant

19. The method according to claim 18, wherein the gel is a hydrogel or a xerogel.

20. A method of preparing a medicament for bone rebuilding and/or treating bone defects, comprising the step of adding osteoclasts to a carrier.

21. The method according to claim 20, wherein the carrier is a suspension medium or a gel or a bone cement or an implant.

22. A medical product or pharmaceutical preparation containing osteoclasts for bone rebuilding.

23. The pharmaceutical preparation or medical product according to claim 22 in the form of a suspension or a gel or a bone cement or an implant.

24. A method for treating a subject, preferably a mammal, having a bone defect, the method comprising the steps of
a) isolating blood from a subject and enriching monocytes,
b) differentiating the monocytes into osteoclasts,
c) administering the osteoclasts obtained in step b) to the subject in or nearby the area of the bone defect.
